(19) United States
(12) Patent Application Publication
(10) Pub. No.: US 2023/0102301 A1
(43) Pub. Date: Mar. 30, 2023

(54) INFORMATION PROCESSING DEVICE, INFORMATION PROCESSING METHOD, AND INFORMATION PROCESSING PROGRAM

(71) Applicant: FUJIFILM Corporation, Tokyo (JP)

(72) Inventor: Haruyasu NAKATSUGAWA, Ashigarakami-gun (JP)

(73) Assignee: FUJIFILM Corporation, Tokyo (JP)

(21) Appl. No.: 17/942,722

(22) Filed: Sep. 12, 2022

(30) Foreign Application Priority Data
Sep. 28, 2021 (JP) 2021-158359

(51) Int. Cl. G16H 50/30

(52) U.S. Cl. CPC G16H 50/30

(57) ABSTRACT

An information processing device includes at least one processor, in which the processor acquires vital information measured over time from the user, and derives a degree of a risk of aggravation of a disease on the basis of an abnormal tendency that appears instantaneously in the vital information.

### CROSS-REFERENCE TO RELATED APPLICATIONS

The present application claims priority under 35 U.S.C. § 119 to Japanese Patent Application No. 2021-158359 filed on Sep. 28, 2021. The above application is hereby expressly incorporated by reference, in its entirety, into the present application.

### BACKGROUND OF THE INVENTION

### 1. Field of the Invention

The present disclosure relates to an information processing device, an information processing method, and an information processing program.

### 2. Description of the Related Art

Conventionally, a technique for estimating the risk of aggravation in a case where a user contracts various diseases is known. For example, JP2020-021509A discloses that aggravation prediction is executed for various diseases (for example, terminal care, diabetic severe hypoglycemia, chronic heart failure, angina pectoris, and myocardial infarction), on the basis of biological information of a user.

Further, for example, JP2019-160015A discloses that the risk of infection with an infectious disease is evaluated according to attribute information such as a user's age, sex, condition (for example, symptoms such as fever), anamnesis, medication history, vaccination history, and test result. In addition, it is disclosed that the risk of infection is made higher as the probability of infection, the probability of onset after infection, and the probability of aggravation increase so that a user who should be examined preferentially is specified and the spread of the outbreak of the infectious disease is restrained.

### SUMMARY OF THE INVENTION

It is known that the new coronavirus infectious disease (COVID-19), which has been prevalent in recent years, has individual differences in the degree of symptoms in a case of being infected, and in particular, a person having underlying diseases (for example, diabetes, and chronic respiratory, heart, kidney, and liver diseases) tends to have a high risk of aggravation. For a user who has been diagnosed as having an underlying disease and is aware of the underlying disease, it can be said that measures against the aggravation of the infectious disease can be taken by thorough prevention of infection by the user himself/herself, priority medical treatment after infection, and the like. In other words, in order to take measures against the aggravation of the infectious disease, it is necessary to diagnose in advance whether or not the user has an underlying disease.

Meanwhile, it is considered that there is a user in the world who is not diagnosed as having an underlying disease but has a sign of becoming the underlying disease (so-called “preliminary group” and “pre-symptomatic state”). In addition, although the user is not diagnosed as having the underlying disease because the user is not in a chronically bad condition, it is considered that there are some users whose conditions worsen temporarily to the same level as that of a person suffering from the underlying disease. Further, it is considered that there are some users who have not been diagnosed because the users are unaware of symptoms even though the users have conditions that should be diagnosed as having the underlying disease. For these users as well, the risk of aggravation of the infectious disease is considered to be comparable to a user who has been diagnosed as having the underlying disease.

In that respect, there is a demand for a technique that can prevent the aggravation of the infectious disease by deriving the risk of aggravation of the infectious disease, in consideration of temporary changes in the condition without prior diagnosis. However, the techniques disclosed in JP2020-021509A and JP2019-160015A cannot derive the risk of aggravation of the infectious disease, in consideration of temporary changes in condition without prior diagnosis.

The present disclosure provides an information processing device, an information processing method, and an information processing program capable of preventing the aggravation of a disease.

According to a first aspect of the present disclosure, there is provided an information processing device comprising: at least one processor, in which the processor acquires vital information measured over time from a user, and derives a degree of a risk of aggravation of a disease on the basis of an abnormal tendency that appears instantaneously in the vital information.

In the first aspect, the disease may be an infectious disease, and the user may be a person who is tested for the infectious disease and who has a positive test result.

In the first aspect, the processor may acquire attribute information indicating an attribute of the user, and may derive the degree of the risk of aggravation on the basis of the vital information and the attribute information.

In the first aspect, the attribute information may indicate at least one of age, sex, or anamnesis of the user.

In the first aspect, the processor may acquire a plurality of different types of vital information measured from the user, and may derive the degree of the risk of aggravation on the basis of the plurality of types of vital information.

In the first aspect, the processor may present, in a case where there is insufficient vital information for deriving the degree of the risk of aggravation, a method of acquiring the insufficient vital information.

In the first aspect, the processor may give a notification of information regarding the user to a device, which is a device other than the information processing device and installed in a medical institution, in a case where the derived degree of the risk of aggravation is a predetermined degree or higher.

In the first aspect, the processor may present a preventive measure for the disease.

In the first aspect, the processor may acquire behavior information indicating behavior of the user, may determine whether or not the user performs a preventive measure for the disease, on the basis of the behavior information, and may issue a warning in a case where the processor determines that the user does not perform the preventive measure for the disease.

In the first aspect, the preventive measure for the disease may depend on a magnitude of the degree of the risk of aggravation derived by the processor.

In the first aspect, the vital information may indicate at least one of a blood glucose level, a blood glucose equivalent level, an electrocardiogram, an arterial blood oxygen saturation, or a blood pressure.

According to a second aspect of the present disclosure, there is provided an information processing method comprising: acquiring vital information measured over time from a user; and deriving a degree of a risk of aggravation of a disease on the basis of an abnormal tendency that appears instantaneously in the vital information.

According to a third aspect of the present disclosure, there is provided an information processing program for causing a computer to execute a process comprising: acquiring vital information measured over time from a user; and deriving a degree of a risk of aggravation of a disease on the basis of an abnormal tendency that appears instantaneously in the vital information.

According to the above aspects, the information processing device, the information processing method, and the information processing program of the present disclosure can prevent the aggravation of a disease.

### BRIEF DESCRIPTION OF THE DRAWINGS

FIG. 1 is a schematic configuration diagram of an information processing system.

FIG. 2 is a block diagram showing an example of a hardware configuration of an information processing device.

FIG. 3 is a block diagram showing an example of a functional configuration of the information processing device.

FIG. 4 is a diagram showing an example of vital information and an abnormal tendency thereof.

FIG. 5 is a diagram illustrating a blood glucose level spike.

FIG. 6 is a diagram illustrating characteristics of HbA1c.

FIG. 7 is a diagram showing an example of a screen displayed on a display.

FIG. 8 is a diagram showing an example of the screen displayed on the display.

FIG. 9 is a diagram showing an example of control corresponding to a degree of a risk of aggravation.

FIG. 10 is a diagram showing an example of control corresponding to the degree of the risk of aggravation.

FIG. 11 is a diagram showing an example of the screen displayed on the display.

FIG. 12 is a flowchart showing an example of update processing in the information processing device.

### DESCRIPTION OF THE PREFERRED EMBODIMENTS

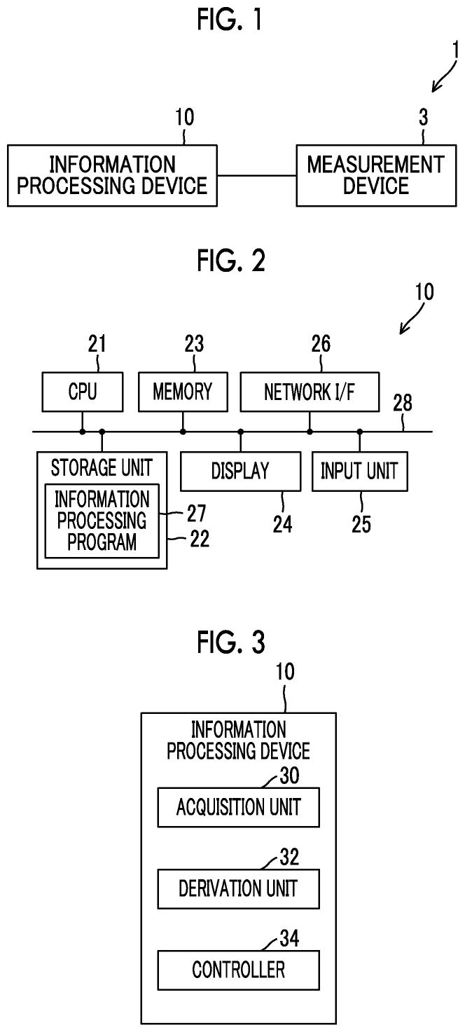

Hereinafter, examples of embodiments of the technique of the present disclosure will be described in detail with reference to the drawings. First, an example of a configuration of an information processing system 1 according to the present embodiment will be described with reference to FIG. 1. As shown in FIG. 1, the information processing system 1 comprises an information processing device 10 and a measurement device 3. The information processing device 10 and the measurement device 3 are connected to each other by wireless or wired communication. As the standard of the wireless communication in this case, for example, Wi-Fi (registered trademark) and Bluetooth (registered trademark) can be appropriately applied.

The measurement device 3 measures at least one type of vital information of a user over time, and transmits the measured vital information to the information processing device 10 by wired or wireless communication. “Measurement over time” means that the vital information is continuously measured at a predetermined time interval. Further, a plurality of the measurement devices 3 that measure different types of vital information may be connected to the information processing device 10.

The vital information is information indicating at least one of a blood glucose level, a blood glucose equivalent level, an electrocardiogram, an arterial blood oxygen saturation (SpO2), or a blood pressure. As the measurement device 3 that measures the blood glucose level, for example, a self-monitoring blood glucose meter using a fingertip puncture method may be applied. The blood glucose equivalent level is vital information that correlates with the blood glucose level, and is, for example, a glucose level in interstitial fluid or blood. As the measurement device 3 that measures the blood glucose equivalent level, for example, a measurement instrument that measures the glucose level in the interstitial fluid as the blood glucose equivalent level by inserting a filament subcutaneously, and a measurement instrument that measures the glucose level in blood as the blood glucose equivalent level by using infrared rays may be applied.

Further, for example, as the measurement device 3, an electrocardiograph that measures an electrocardiogram, a pulse oximeter that measures SpO2, and a sphygmomanometer that measures a blood pressure may be applied. Further, for example, as the measurement device 3, a wearable terminal, such as a smartwatch provided with a sensor that measures various types of vital information, may be applied.

The information processing device 10 derives the degree of the risk of aggravation of the disease on the basis of the abnormal tendency of the vital information measured by the measurement device 3. In the following examples of the embodiments, a description will be given by using the new coronavirus infectious disease (COVID-19) as an example of the disease, but the technique of the present disclosure can be applied to other infectious diseases (for example, influenza virus infection diseases) and various diseases other than the infectious disease. Hereinafter, the configuration and the function of the information processing device 10 will be described.

First, an example of a hardware configuration of the information processing device 10 according to the present embodiment will be described with reference to FIG. 2. As shown in FIG. 2, the information processing device 10 includes a CPU 21, a non-volatile storage unit 22, and a memory 23 serving as a temporary storage area. In addition, the information processing device 10 includes a display 24 such as a liquid crystal display, an input unit 25 such as a keyboard, a mouse, and a touch panel, and a network interface (I/F) 26. The network I/F 26 performs wired or wireless communication with the measurement device 3 and an external network (not shown). The CPU 21, the storage unit 22, the memory 23, the display 24, the input unit 25, and the network I/F 26 are connected to each other via a bus 28, such as a system bus and a control bus, so that various types of information can be exchanged.

The storage unit 22 is realized by, for example, a storage medium such as a hard disk drive (HDD), a solid state drive (SSD), and a flash memory. An information processing program 27 in the information processing device 10 is stored in the storage unit 22. The CPU 21 reads out the information processing program 27 from the storage unit 22 and then develops the information processing program 27 into the memory 23, and executes the developed information processing program 27. The CPU 21 is an example of a processor of the present disclosure. As the information processing device 10, for example, a smartphone, a tablet terminal, a wearable terminal, a personal computer, or a server computer can be appropriately applied.

Next, an example of a functional configuration of the information processing device 10 according to the present embodiment will be described with reference to FIG. 3. As shown in FIG. 3, the information processing device 10 includes an acquisition unit 30, a derivation unit 32, and a controller 34. The CPU 21 executes the information processing program 27, whereby the CPU 21 functions as the acquisition unit 30, the derivation unit 32, and the controller 34.

The acquisition unit 30 acquires the vital information measured over time from the user by the measurement device 3, from the measurement device 3. The derivation unit 32 derives the degree of the risk of aggravation of the disease on the basis of the abnormal tendency that appears instantaneously in the vital information acquired by the acquisition unit 30. “Instantaneously appearing abnormal tendency” refers to a phenomenon in which vital information is basically normal, but an abnormality is temporarily observed in the vital information. That is, since the period during which the vital information is normal is long, it is necessary to monitor the vital information over time in order to detect the abnormality.

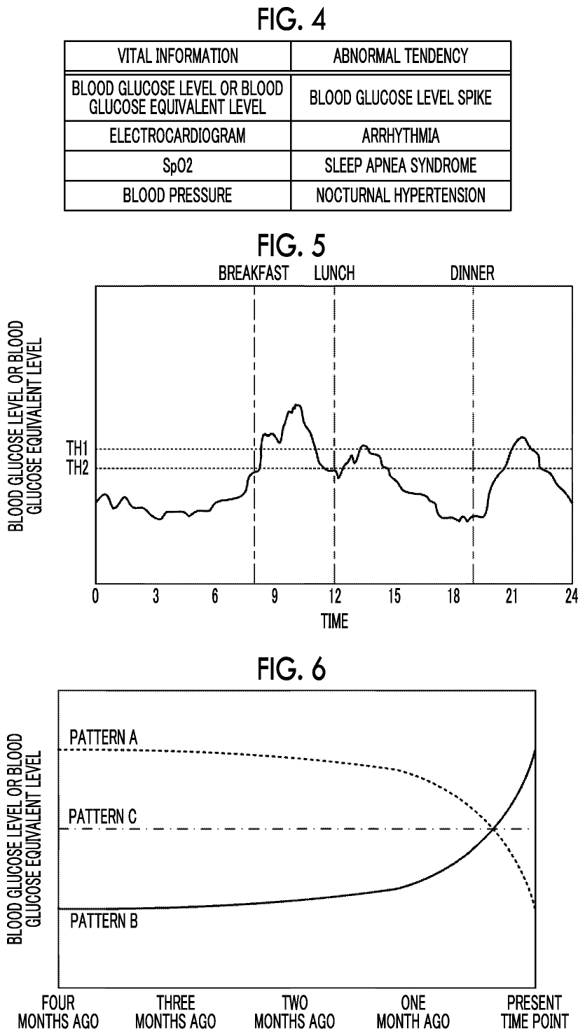

FIG. 4 shows an example of the vital information and the abnormal tendency thereof. Hereinafter, a specific example of a method of deriving the degree of the risk of aggravation corresponding to the abnormal tendency that appears instantaneously for each vital information shown in FIG. 4 will be described.

### Blood Glucose Level or Blood Glucose Equivalent Level

As shown in FIG. 4, an example of the abnormal tendency that appears instantaneously in the blood glucose level or the blood glucose equivalent level includes a blood glucose level spike. FIG. 5 shows a diagram showing intra-day fluctuations in blood glucose level or blood glucose equivalent level in which a blood glucose level spike is observed. “Blood glucose level spike” is a symptom observed in the preliminary group of the diabetes, and is a symptom in which the blood glucose level sharply rises and sharply drops temporarily about 1 to 2 hours after a meal even in a case where the fasting blood glucose level is within the normal range.

The derivation unit 32 may determine that a severe blood glucose level spike is observed in a case where the blood glucose level or the blood glucose equivalent level acquired by the acquisition unit 30 temporarily exceeds a predetermined threshold value TH1, and may derive the risk of aggravation to be high. On the other hand, the derivation unit 32 may determine that a mild blood glucose level spike is observed in a case where the blood glucose level or the blood glucose equivalent level acquired by the acquisition unit 30 does not exceed the threshold value TH1 but temporarily exceeds a predetermined threshold value TH2 (note that TH1>TH2), and may derive the risk of aggravation to be moderate. In the example of FIG. 5, since the blood glucose level or the blood glucose equivalent level temporarily exceeds the predetermined threshold value TH1, the derivation unit 32 derives the risk of aggravation to be high.

In addition, FIG. 6 shows a diagram showing long-term fluctuations in blood glucose level or blood glucose equivalent level from the present time point to four months ago. Generally, in the diagnosis of diabetes, the blood glucose level and an HbA1c value obtained by a blood test are used. HbA1c is a value representing a weighted average of blood glucose levels over the past several months, which has a greater weight as it approaches the present time point, and is a value that does not depend on intra-day fluctuations in blood glucose level as shown in FIG. 5. However, in HbA1c, a pattern A (shown by a dotted line) in which the latest blood glucose level sharply drops, a pattern B (shown by a solid line) in which the latest blood glucose level sharply rises, and a pattern C (shown by an alternating long-dash and short-dash line) in which there is no fluctuation in blood glucose level may have the same values, as shown in FIG. 6. That is, with HbA1c, it is difficult to accurately grasp the fluctuation tendency of the blood glucose level or the blood glucose equivalent level.

In that respect, the derivation unit 32 may determine that the blood glucose level tends to be improved in a case where the blood glucose level or the blood glucose equivalent level acquired by the acquisition unit 30 over time tends to sharply drop in the pattern A, and may derive the risk of aggravation to be low. On the other hand, the derivation unit 32 may determine that the blood glucose level tends to worsen in a case where the blood glucose level or the blood glucose equivalent level acquired by the acquisition unit 30 tends to sharply rise in the pattern B, and may derive the risk of aggravation to be high.

### Electrocardiogram

As shown in FIG. 4, an example of the abnormal tendency that appears instantaneously on an electrocardiogram includes arrhythmia (for example, atrial fibrillation and atrial flutter). It is known that arrhythmia is one type of the underlying diseases of COVID-19, and there is a high probability of developing a heart disease in a case of contracting COVID-19. In that respect, the derivation unit 32 may derive the risk of aggravation to be high in a case where the electrocardiogram acquired by the acquisition unit 30 shows a movement indicating arrhythmia.

### SpO2

As shown in FIG. 4, an example of the abnormal tendency that appears instantaneously in SpO2 is sleep apnea syndrome. It is known that sleep apnea syndrome is one type of the underlying diseases of COVID-19, and there is a high probability of developing respiratory failure in a case of contracting COVID-19. The value of SpO2 decreases in the apnea state. In that respect, the derivation unit 32 may determine that the tendency of sleep apnea syndrome is observed in a case where the sleep SpO2 acquired by the acquisition unit 30 is a predetermined threshold value or less, and may derive the risk of aggravation to be high.

### Blood Pressure

As shown in FIG. 4, an example of the abnormal tendency that appears instantaneously in blood pressure includes nocturnal hypertension. It is known that a user with nocturnal hypertension has a high probability of vascular endothelial and organ disorders and has a probability of developing a cardiovascular disease in a case of contracting COVID-19. In that respect, the derivation unit 32 may determine that the tendency of nocturnal hypertension is observed in a case where the nocturnal blood pressure acquired by the acquisition unit 30 is a predetermined threshold value or more, and may derive the risk of aggravation to be high.

Further, the derivation unit 32 may derive the degree of the risk of aggravation by using a plurality of types of vital information in a complex manner. Specifically, the acquisition unit 30 acquires a plurality of different types of vital information measured from the user, from the measurement devices 3. The derivation unit 32 derives the degree of the risk of aggravation on the basis of the plurality of types of vital information acquired by the acquisition unit 30. For example, the derivation unit 32 may derive the risk of aggravation to be low in a case where a mild abnormal tendency is observed in only one type of vital information, and may derive the risk of aggravation to be high in a case where a mild abnormal tendency is observed in the plurality of types of vital information.

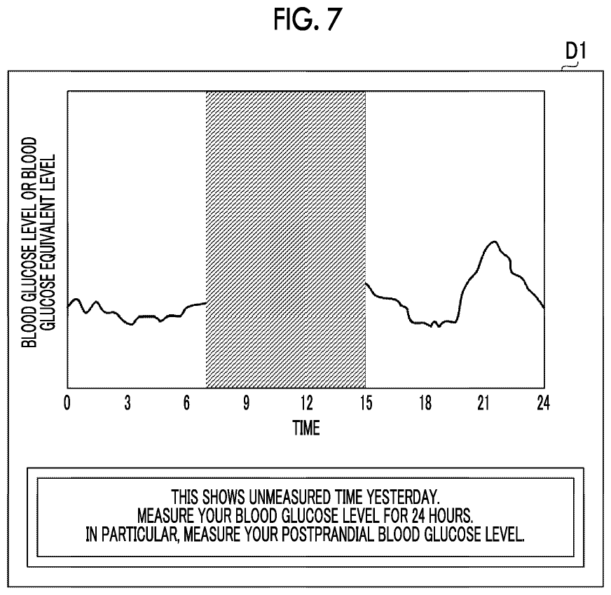

In a case where there is insufficient vital information for deriving the degree of the risk of aggravation, the controller 34 may present a method of acquiring the insufficient vital information. FIG. 7 shows an example of a screen D1 in which the period during which the vital information should be acquired is shown, as an example of the method of acquiring the vital information. The screen D1 is a screen displayed on a display 24 by the controller 34. As shown in FIG. 7, in a case where a part of the vital information measured over time is missing, the controller 34 may give a notification of the fact and may give attention not to miss the vital information.

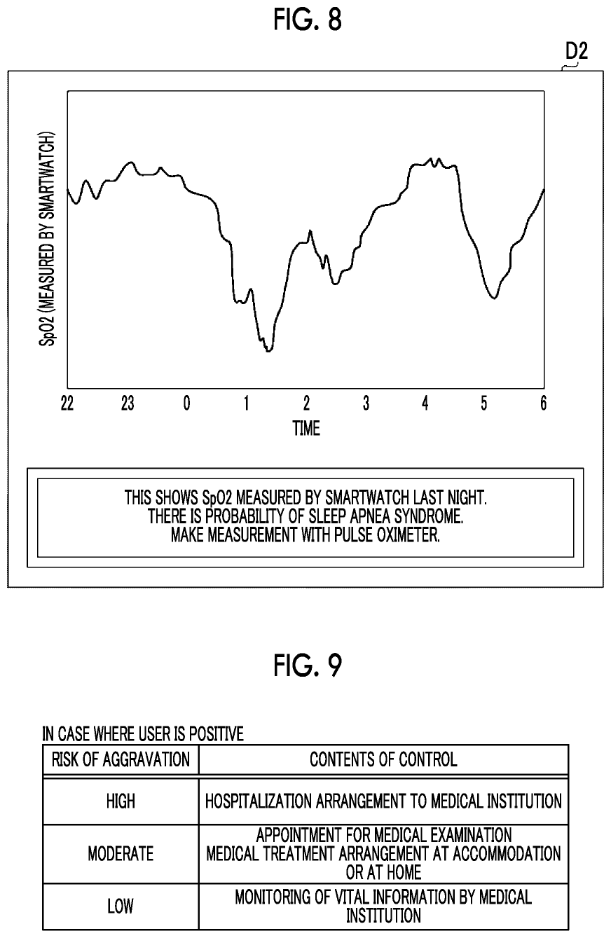

FIG. 8 shows an example of a screen D2 showing the designation of the measurement device 3 to acquire the vital information, as an example of the method of acquiring the vital information. The screen D2 is a screen displayed on the display 24 by the controller 34. As the measurement device 3 for SpO2, a wearable terminal such as a smartwatch provided with a sensor that measures the oxygen concentration in blood by using reflected light can be used. However, the measurement accuracy of SpO2 performed by the wearable terminal is lower than that of the pulse oximeter that measures the oxygen concentration in blood by using transmitted light. In that respect, for example, as shown in FIG. 8, the derivation unit 32 may derive that there is a probability of sleep apnea syndrome (that is, there is the risk of aggravation) on the basis of SpO2 measured by the wearable terminal, and then the controller 34 may recommend acquiring the SpO2 using the pulse oximeter.

In addition, the controller 34 performs various types of control according to the derived degree of the risk of aggravation. FIG. 9 shows the control contents corresponding to the degree of the risk of aggravation in a case where the user is a person who is already tested for the infectious disease and who has a positive test result. In a case where the user is already infected with the infectious disease, it is preferable to prevent the aggravation by taking appropriate measures corresponding to the risk of aggravation. In that respect, as shown in FIG. 9, the controller 34 may cooperate with a medical institution, such as a hospitalization arrangement or a medical treatment arrangement, a vital information monitoring instruction, and an appointment for a medical examination. That is, the controller 34 may give a notification of information regarding the user to a device, which is a device other than the information processing device 10 and installed in the medical institution, in a case where the derived degree of the risk of aggravation is a predetermined degree or higher. According to such an aspect, the medical institution can grasp the user who has a high risk of aggravation and who should be treated with priority, so that the aggravation can be effectively prevented.

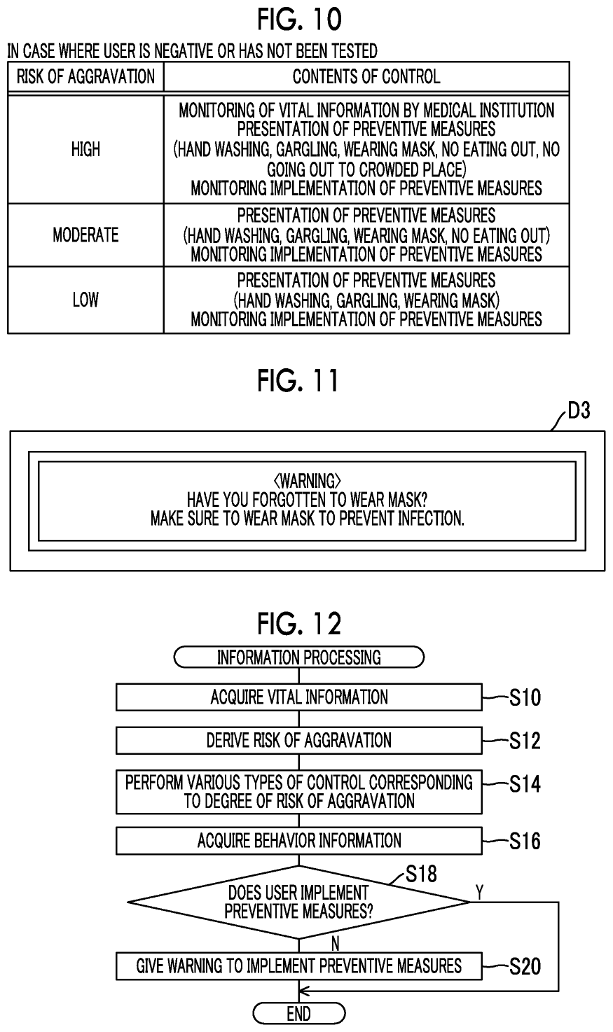

FIG. 10 shows the control contents corresponding to the degree of the risk of aggravation in a case where the user is a person who is already tested for the infectious disease and who has a negative test result or in a case where the user has not been tested for the infectious disease. For example, the controller 34 may instruct the medical institution to monitor the vital information so that a user at a high risk of aggravation can be treated promptly at the time of infection. Further, for example, as shown in FIG. 10, the controller 34 may present a preventive measure for the disease. In this case, the preventive measure for the disease may depend on the magnitude of the degree of the risk of aggravation. The contents of various types of control corresponding to the degree of the risk of aggravation, which are shown in FIGS. 9 and 10, are stored in advance in, for example, the storage unit 22.

Further, the controller 34 may monitor whether the user implements the preventive measure for the disease. Specifically, the controller 34 acquires behavior information indicating the behavior of the user, and determines whether or not the user performs the preventive measure for the disease, on the basis of the behavior information. The behavior information is, for example, information indicating the position of the user, information indicating the motion obtained by a sensor such as an acceleration sensor and a gyro sensor, and information indicating whether or not a mask is worn, which is obtained by analyzing a moving image captured by a camera. For example, the controller 34 may determine whether the user has gone to a restaurant or a crowded place, on the basis of the information indicating the position of the user. Further, for example, the controller 34 may determine whether the user has performed hand washing and gargling, on the basis of the information indicating the motion, which is obtained by the sensor.

Further, the controller 34 may perform control to issue a warning in a case where the controller 34 determines that the user does not perform the preventive measure for the disease. FIG. 11 shows an example of a screen D3 displayed on the display 24 in a case where the user does not wear a mask as an example of the preventive measure for the disease. In this way, it is possible to contribute to the prevention of the infection spread of the infectious disease and the prevention of the aggravation by monitoring whether or not the user performs the preventive measure appropriately and issuing a warning in a case where the user does not perform the preventive measure.

Next, an action of the information processing device 10 according to the present embodiment will be described with reference to FIG. 12. In the information processing device 10, the CPU 21 executes the information processing program 27, whereby information processing shown in FIG. 12 is executed. The information processing is executed, for example, in a case where the user gives an instruction to start execution via the input unit 25.

In step S10, the acquisition unit 30 acquires the vital information measured over time from the user by the measurement device 3, from the measurement device 3. In step S12, the derivation unit 32 derives the degree of the risk of aggravation of the disease on the basis of the abnormal tendency that appears instantaneously in the vital information acquired in step S10. In step S14, the controller 34 performs various types of control corresponding to the degree of the risk of aggravation derived in step S12 (for example, notification of information regarding the user with respect to a device, which is a device other than the information processing device 10 and installed in the medical institution, and the presentation of the preventive measure for the disease corresponding to the magnitude of the degree of the risk of aggravation).

In step S16, the controller 34 acquires the behavior information indicating the behavior of the user. In step S18, the controller 34 determines whether or not the user performs the preventive measure for the disease on the basis of the behavior information acquired in step S16. In a case where the controller 34 determines that the user does not perform the preventive measure for the disease (that is, in a case where step S18 is N), in step S20, the controller 34 performs control to issue a warning to perform the preventive measure for the disease, and ends this information processing. On the other hand, in a case where the controller 34 determines that the user performs the preventive measure for the disease (that is, in a case where step S18 is Y), the controller 34 does not perform the processing of step S20 and ends this information processing as it is.

As described above, the information processing device 10 according to a preferred aspect of the present disclosure comprises at least one processor, and the processor acquires the vital information measured over time from the user, and derives the degree of the risk of aggravation of the disease on the basis of the abnormal tendency that appears instantaneously in the vital information. That is, with the information processing device 10 according to the present embodiment, the risk of aggravation of the disease can be derived in consideration of a temporary change in the condition without prior diagnosis, so that the aggravation of the disease can be prevented.

In the above embodiment, the information processing device 10 may make the methods of deriving the degree of the risk of aggravation different from each other in consideration of the attribute of the user. Specifically, the acquisition unit 30 may acquire attribute information indicating the attribute of the user. The attribute information is, for example, information indicating at least one of the age, sex, or anamnesis of the user. For example, the acquisition unit 30 may acquire the attribute information input by the user via the input unit 25, or may acquire the attribute information via the network from an electronic medical record managed by an external management server (not shown) installed in the medical institution or the like.

The derivation unit 32 may derive the degree of the risk of aggravation on the basis of the vital information and the attribute information. For example, the derivation unit 32 may determine whether or not the vital information has the abnormal tendency by using a plurality of predetermined threshold values that are different for each attribute information. That is, the derivation unit 32 may make the easiness of determining the abnormal tendency different for each user by making the threshold values for determining the abnormal tendency different for each attribute information. For example, with regard to COVID-19, it is known that elderly people have a higher risk of aggravation than young people, men have a higher risk of aggravation than women, and those who have anamnesis (for example, an underlying disease) have a higher risk of aggravation than those who do not have anamnesis. It is possible to derive the risk of aggravation more appropriately by making the methods of deriving the degree of the risk of aggravation different according to these attributes.

Further, in the above embodiment, the aspect (see FIGS. 9 and 10) in which the degree of the risk of aggravation is expressed in three stages, that is, high, moderate, and low, has been described, but the present disclosure is not limited thereto. The degree of the risk of aggravation may be expressed numerically, for example.

In each of the above embodiments, for example, the following various processors can be used as the hardware structure of a processing unit that executes various types of processing, such as the acquisition unit 30, the derivation unit 32, and the controller 34. The above-described various processors include, for example, a programmable logic device (PLD) which is a processor having a changeable circuit configuration after manufacture, such as a field programmable gate array (FPGA), and a dedicated electrical circuit which is a processor having a dedicated circuit configuration designed to perform specific processing, such as an application specific integrated circuit (ASIC), in addition to the CPU which is a general-purpose processor that executes software (programs) to function as various processing units, as described above.

One processing unit may be composed of one of these various processors or a combination of two or more processors of the same type or different types (for example, a combination of a plurality of FPGAs or a combination of a CPU and an FPGA). Alternatively, a plurality of processing units may be composed of one processor.

A first example in which a plurality of processing units are composed of one processor is an aspect in which one or more CPUs and software are combined to constitute one processor and the processor functions as the plurality of processing units, as typified by a computer, such as a client and a server. A second example is an aspect in which a processor that realizes all the functions of a system including the plurality of processing units with one integrated circuit (IC) chip is used, as typified by a system on chip (SoC). As described above, various processing units are formed of one or more of the above-described various processors as the hardware structure.

Further, as the hardware structure of these various processors, more specifically, an electric circuit (circuitry) in which circuit elements, such as semiconductor elements, are combined can be used.

Further, in each of the above embodiments, the aspect in which the information processing program 27 is stored (installed) in the storage unit 22 in advance has been described, but the present disclosure is not limited thereto. The information processing program 27 may be provided in a form of being recorded on a recording medium, such as a compact disc read only memory (CD-ROM), a digital versatile disc read only memory (DVD-ROM), and a Universal Serial Bus (USB) memory. Alternatively, the information processing program 27 may be downloaded from an external device via the network. Furthermore, the technique of the present disclosure extends to a storage medium on which the information processing program is non-temporarily stored, in addition to the information processing program.

In the technique of the present disclosure, the above examples of embodiments can be appropriately combined with each other. The contents described and shown above are detailed descriptions of the parts related to the technique of the present disclosure, and are merely an example of the technique of the present disclosure. For example, the descriptions for the above configurations, functions, operations, and effects are the descriptions for an example of the configurations, functions, operations, and effects of parts related to the technique of the present disclosure. Accordingly, it goes without saying that an unnecessary part may be deleted, a new element may be added, or replacement may be made with respect to the contents described and shown above, within the scope not departing from the gist of the technique of the present disclosure.

### EXPLANATION OF REFERENCES

1: information processing system

3: measurement device

10: information processing device

21: CPU

22: storage unit

23: memory

24: display

25: input unit

26: network I/F

27: information processing program

28: bus

30: acquisition unit

32: derivation unit

34: controller

D1 to D3: screen

What is claimed is:

1. An information processing device comprising:
at least one processor,
wherein the processor
acquires vital information measured over time from a user, and
derives a degree of a risk of aggravation of a disease on the basis of an abnormal tendency that appears instantaneously in the vital information.

2. The information processing device according to claim 1,
wherein the disease is an infectious disease, and
the user is a person who is tested for the infectious disease and who has a positive test result.

3. The information processing device according to claim 1,
wherein the processor
acquires attribute information indicating an attribute of the user, and
derives the degree of the risk of aggravation on the basis of the vital information and the attribute information.

4. The information processing device according to claim 3,
wherein the attribute information indicates at least one of age, sex, or anamnesis of the user.

5. The information processing device according to claim 1,
wherein the processor
acquires a plurality of different types of vital information measured from the user, and
derives the degree of the risk of aggravation on the basis of the plurality of types of vital information.

6. The information processing device according to claim 1,
wherein the processor
presents, in a case where there is insufficient vital information for deriving the degree of the risk of aggravation, a method of acquiring the insufficient vital information.

7. The information processing device according to claim 1,
wherein the processor
gives a notification of information regarding the user to a device, which is a device other than the information processing device and installed in a medical institution, in a case where the derived degree of the risk of aggravation is a predetermined degree or higher.

8. The information processing device according to claim 1,
wherein the processor
presents a preventive measure for the disease.

9. The information processing device according to claim 1,
wherein the processor
acquires behavior information indicating behavior of the user,
determines whether or not the user performs a preventive measure for the disease, on the basis of the behavior information, and
issues a warning in a case where the processor determines that the user does not perform the preventive measure for the disease.

10. The information processing device according to claim 8,
wherein the preventive measure for the disease depends on a magnitude of the degree of the risk of aggravation derived by the processor.

11. The information processing device according to claim 9,
wherein the preventive measure for the disease depends on a magnitude of the degree of the risk of aggravation derived by the processor.

12. The information processing device according to claim 1,
wherein the vital information indicates at least one of a blood glucose level, a blood glucose equivalent level, an electrocardiogram, an arterial blood oxygen saturation, or a blood pressure.

13. An information processing method comprising:
acquiring vital information measured over time from a user; and
deriving a degree of a risk of aggravation of a disease on the basis of an abnormal tendency that appears instantaneously in the vital information.

14. A non-transitory computer-readable storage medium storing an information processing program for causing a computer to execute a process comprising:
acquiring vital information measured over time from a user; and
deriving a degree of a risk of aggravation of a disease on the basis of an abnormal tendency that appears instantaneously in the vital information.
